(19) United States
(12) Patent Application Publication
(10) Pub. No.: US 2025/0138220 A1
(43) Pub. Date: May 1, 2025

(54) Real Time and Autonomous Petrophysical Formation Evaluation and Machine Learning Deployment

(71) Applicant: Saudi Arabian Oil Company, Dhahran (SA)

(72) Inventors: Lautaro Rayo, Dhahran (SA); Majed Fareed Kanfar, Dammam (SA); Abdullah A. Alakeely, Dhahran (SA); Vladislav Torlov, Dhahran (SA); Yacine Meridji, Dhahran (SA)

(21) Appl. No.: 18/497,557

(22) Filed: Oct. 30, 2023

(51) Int. Cl. G01V 20/00; G16C 20/70; G16C 20/80

(52) U.S. Cl. CPC G01V 20/00; G16C 20/70; G16C 20/80

(57) ABSTRACT

A computer implemented method is described. The method includes streaming data comprising petrophysical data associated with at least one subsurface formation obtained in real time. The method includes analyzing the stream of data to determine at least one model configured to evaluate the at least one subsurface formation. The method includes executing the at least one model to evaluate the at least one subsurface formation using the stream of data as input. Additionally, the method includes outputting a representation of formation characteristics in real time.

### TECHNICAL FIELD

This disclosure relates generally to autonomous petrophysical formation evaluation and machine learning deployment.

### BACKGROUND

During drilling, data associated with subsurface formations is obtained. The data captured during drilling can vary in format based on a number of factors. The data is often unusable until manual interpretation of the data is performed. Partially-informed or uninformed decisions are made based on partial or absent analysis of data at the time the decision has to be made.

### BRIEF DESCRIPTION OF DRAWINGS

FIG. 1 shows a workflow that enables real-time and autonomous petrophysical formation evaluation and machine learning deployment.

FIG. 2 shows a workflow between a State Store System and a Data Consultation and Extraction System.

FIG. 3 is a process flow diagram of a process that enables real-time and autonomous petrophysical formation evaluation and machine learning deployment.

FIG. 4 illustrates hydrocarbon production operations that include both one or more field operations and one or more computational operations, which exchange information and control exploration for the production of hydrocarbons.

FIG. 5 is a schematic illustration of an example controller (or control system) for that enables real time and autonomous petrophysical formation evaluation and machine learning deployment.

### DETAILED DESCRIPTION

A real time and autonomous petrophysical formation evaluation and machine learning deployment is described. The present systems and techniques include an autonomous petrophysics (AP) platform. The autonomous petrophysics platform executes according to a workflow that enables the analysis of petrophysical data and drilling data as it is acquired. The present systems and techniques enable real-time and autonomous petrophysical formation evaluation associated with multiple wellbores simultaneously. In examples, visualizations of wellbores are generated in real time and a user provides feedback for any number of wellbores simultaneously. The present systems and techniques autonomously detect data arriving from new, unseen wellbores, and automatically adds the new, unseen wellbores to a stack of assets availed to the user. In examples, the present techniques store acquired petrophysical data and drilling data, and also provide remote access to the petrophysical data and drilling data over a network. The data is transformed to obtain a representation of the formation characteristics. In some examples, visualizations are automatically generated whenever updated petrophysical data and drilling data is stored. Additionally, the updated data is deployed to users. Thus, the present techniques to enable users to share information in real time in a to derive formation characteristics regardless of the format of the obtained data. The present techniques eliminate costs, risks and opportunity-losses related to waiting for a manual analysis to be performed on captured data.

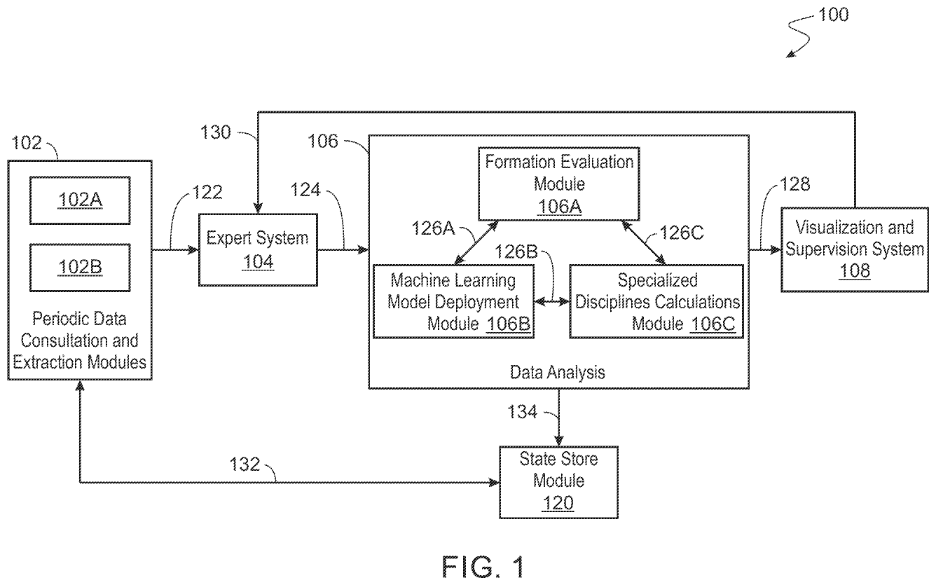

FIG. 1 shows a workflow 100 that enables real-time and autonomous petrophysical formation evaluation and machine learning deployment. The workflow is described according to the communications and functions of various systems of the autonomous petrophysical platform. Software, hardware, or any combinations thereof that implement an autonomous petrophysical platform includes, for example, the workflow and systems described with respect to FIG. 1. Table 1 lists the systems described with respect to FIG. 1, followed in parenthesis with exemplary software that implements each one of these components.

TABLE 1
AP Components

| 1. Data Consultation and Extraction | |
|---|---|
| a. | WITSML-Client (WITConnect) |
| b. | Corporate-DB (OracleConnect) |
| 2. Expert System (ExpSys) | |
| 3. Data Analysis System | |
| a. | Formation Analysis (logSolver) |
| b. | Machine Learning Deployment (Custom |
| | Deployer) |
| c. | Specialized Calculations (Physical Models) |

4. State Store System (Local Database)
5. Visualization and Supervision System (WebApp)

Although particular software packages are listed in Table 1, the present techniques are not limited to the software listed. The present techniques are enabled according to the workflow 100 described with respect to FIG. 1.

In the example of FIG. 1, a data consultation and extraction system (DC&ES) 102 is shown. The DC&ES 102 is used to obtain petrophysical data and drilling data. In some embodiments, petrophysical data and drilling data are evaluated as the is acquired, in real time. Petrophysical data and drilling data is acquired continuously as wellbores are drilled. The present systems and techniques connect to databases that collect petrophysical data and drilling data in real time.

In examples, the DC&ES 102 extracts data from a number of databases. The DC&ES 102 obtains data from at least one database that hosts data pushed to it in real-time, periodically, or at frequent intervals. The pushed data is streaming data that is generated continuously by one or more data sources. In examples, the petrophysical data is streaming data including a continuous measurement of formation properties with electrically powered instruments to infer properties and make decisions about drilling and production operations. In drilling data is streaming data associated with electrically powered instruments, tools, and other infrastructure associated with a drilling operations at a respective wellbore.

In the example of FIG. 1, the DC&ES 102 obtains data from state store system 120. The petrophysical data and drilling data is stored in a state store system 120. Arrow 132 shows the flow of data from the periodic data consultation and extraction modules 102 to the state store system 120. The state store system 120 also stores data output by a Data Analysis System (DAS) 106. Arrow 134 shows the flow of data from the DAS 106 to the state store system 120.

In examples, the state store system 120 is a database with high update frequency and contains the bulk of the volume of data to be analyzed. This data is comprised of the actual petrophysical and drilling measurements that are made continuously as the drilling operation progresses. Other databases with lower update frequency are consulted to obtain contextual information used by an Expert System (ES) 104 provide accurate analysis parameters to the DAS 106.

In examples, an implementation of the DC&ES 102 is achieved by hosting data using an application 102A based on a Well-Site Information Transfer Standard Markup Language (WITSML) standard, such as WITSML Version 2.0 was released in February 2017 by Energistics. For example, the application 102A is a Python client for servers hosting data.

In examples, an implementation of the DC&ES 102 is achieved by hosting data using an application 102B based on a relational database management system (RDBMS), built using a structure query language. For example, the application 102B is a Python client for Oracle servers hosting data. The application 102B extracts wellbore-related metadata that serves as contextual input to the ES 104. The ES 104 uses the context data to derive parameters used to evaluate data originating from any given wellbore.

In the example of FIG. 1, the petrophysical data and drilling data is continuously analyzed using the DAS 106 as the data continues to be collected during the drilling operation. The analysis of this data occurs autonomously, as the parameters used to perform this analysis are deduced by an ES 104. Arrow 122 shows the flow of data from the periodic data consultation and extraction modules 102 to the ES 104. The ES 104 formulates these parameters from the data itself and from metadata collected from several databases. In examples, the parameters derived by the ES 104 come in the form of specific pre-defined models corresponding to each of the DAS sub-systems 126A, 126B, and 126C. The DAS 106 receives the specific pre-defined models and executes the models to autonomously evaluate a petrophysical formation.

The workflow analyses the data at the DAS 106 based on the parameters derived by the ES 104 and data from the periodic data consultation and extraction modules 102. Arrow 124 shows the flow of data from the ES 104 to the DAS 106.

The DAS 106 is composed of three sub-systems: formation analysis subsystem 106A, machine learning model deployment subsystem 106B, and specialized calculations subsystem 106C. The three sub-systems are communicatively coupled as shown by arrows 126A, 126B, and 126C. The formation evaluation sub-system 106A obtains as input the measurements (Density, Gamma Ray, Resistivity, . . . ) and at least one petrophysical model to generate an output. The present systems and techniques automatically prescribe these petrophysical models required for the analysis (and the input measurements in real time). The workflow 100 contains a visualization and supervision system (VSS) 108 to continuously display the collected data and its associated analysis. Arrow 128 shows the flow of data from the DAS 106 to the VSS system 108. VSS system 108 serves to collect feedback from the user, in a form that enables the user to improve upon the parameters deduced by the autonomous system. The parameters inputted in this manner are obtained in the ES 104 and are enforced for the rest of the real-time, autonomous evaluation. Arrow 130 shows the flow of data from the VSS 108 to the ES 104.

The ES 104 provides autonomy to the autonomous petrophysical platform. The ES 104 enables the autonomous petrophysical platform to provide petrophysical solutions without manual interpretation. The ES 104 maps any wellbore streaming real-time petrophysical data to a set of instructions needed for full evaluation of the respective wellbore. The instructions are: (i) Formation Analysis Model Parameters (corresponding to the formation evaluation subsystem 106A of FIG. 1); (ii) Machine Learning Models to be Deployed and their parameters (corresponding to the machine learning deployment subsystem 106B of FIG. 1); and (iii) Specialized Calculations and their parameters (corresponding to the specialized disciplines calculation subsystem 106C of FIG. 1).

In some embodiments, formation analysis at the formation evaluation subsystem 106A is performed by a petrophysical solver, such as a Petrophysical Solver shown in Table 1. In examples, a petrophysical solver is an application that generates robust subsurface characteristics. The petrophysical solver obtains the raw petrophysical measurements extracted by an application 102A, such as a WITSML-client (Table 1) as wells as a series of physical parameters. The physical parameters provide context to the petrophysical solver. A fully defined set of petrophysical parameters is called a petrophysical model. In examples, petrophysical models are predefined using industry standard workflows. The petrophysical models are stored (e.g., such as stored on disk or cloud storage). In examples, metadata associated with the physical models is stored and includes a context for each respective petrophysical model. For example, the context describes conditions under which a petrophysical model is designed to be deployed.

In examples, a petrophysical model includes metadata that describes the set of measurements it accepts as input. For example, these measurements include but are not limited to Density, Neutron Porosity, Gamma Ray. The measurements depend on the particular tool used to capture downhole data at the wellbore.

In examples, a petrophysical model includes metadata that describes the which physics to choose for the description of each measurement. This depends both on the technology of the specific tool making the specific measurement but also on decisions about what physics best describe the specific formation. Examples of physics to select include Archie Equation and its parameters, Dual-Water Equation and its parameters, Specific Neutron Tool physics.

In examples, a petrophysical model includes metadata that describes the uncertainty associated to each measurement at each depth. This depends on the specific measurement and in borehole conditions.

In examples, a petrophysical model includes metadata that describes which set of volumes it aims to solve for. Example of sets of volumes include Calcite, Dolomite, Anhydrite, Water, Oil. This depends on the geology of the formation transected by the borehole. Additionally, in examples, a petrophysical model includes metadata that describes the properties of the minerals and fluids in question. Examples of the properties include Oil API, Formation Temperature and Pressure. In examples, a petrophysical model includes metadata that describes the properties of the borehole environment. Example: Mud Parameters.

In examples, the petrophysical models are structured objects that specify all aforementioned parameters, in a format that can be decoded by the formation evaluation software (for example, logSolver).

In examples, the context is a structured object that summarizes metadata about the wellbore being analyzed. Many of the context parameters are parameters that the petrophysical model directly uses, for example, the context includes information that identifies which tool is in the hole, and therefore which measurements the model should accept and which uncertainty to prescribe to such measurements. Another example of context directly influencing the petrophysical model is Mud Type/Parameters. The context includes which mud (oil based mud, water based mud) is in the system, and what are its parameters: Density, Salinity.

In examples, context also includes parameters that must pass by an indirect process for them to influence actual values to the underlying petrophysical model. For example, the context includes the name of the formation the borehole is currently intersecting, but that information must be processed by the ES 104 in order to associate specific volumes to solve for to that formation name. For example, the context includes information that indicates the location of drilling, such as drilling the Arab-D formation, but it is the Expert sub-system that translates that information into actual volumes to solve for: Calcite, Water, Oil & Porosity in this example.

In examples, the autonomous platform defines context as the value of the parameters in Table 2.

TABLE 2
Context Definition

| | |
|---|---|
| 1. | Target Subsurface Unit: Geological/Petrophysical/Engineering Sub-Zone the |
| | model is intended to be deployed across |
| 2. | Target Hydrocarbon: The hydrocarbon type (Oil/Gas) the model is intended |
| | to solve for |
| 3. | Mud Type/Mud Parameters: The mud system the wellbore is being drilled |
| | with |
| 4. | Bit Size: The size of the drilling bit being utilized to drill the wellbore |
| 5. | Service Company: The service company acquiring the petrophysical data |

In examples, the context is formed from a combination of subsurface unit, hydrocarbon type, mud type, bit size and service company. The ES 104 deduces context from the available data and ultimately decides on which Petrophysical Model to deploy based on the associated context provided.

A set of instructions generated by the ES 104 for full evaluation of the respective wellbore also includes machine learning models to be deployed. In examples, the autonomous platform enables the deployment of machine learning models. In examples, the machine learning models are trained and stored on disk or at a cloud storage location prior to deployment. The machine learning models are associated with metadata that specifies the inputs are executed by the machine learning model, the outputs the machine learning model generates, and the context under which the machine learning models are to be deployed.

In examples, the inputs are used to determine if the machine learning model is deployed. For example, when the petrophysical data and drilling data includes data types corresponding to the inputs of a particular machine learning model, the particular machine learning model is deployed. The ES 104 derives context from the available data and ultimately decides on which machine learning model(s) to deploy based on the associated context provided.

A set of instructions generated by the ES 104 for full evaluation of the respective wellbore also includes specialized calculations. The autonomous platform enables the real-time deployment of any type of calculation or analytical model. These calculations or models are previously defined and stored together with the context described in Table 2 in which they should be deployed. Specific parameters input to the specialized calculations are also stored with the respective specialized calculations. In examples, the metadata associated with specialized calculations specifies how specific parameters are constructed from raw petrophysical/drilling parameters. The way these parameters are constructed is varies depending on context as described with respect to Table 2.

In some embodiments, the ES 104 is a mapping tool that prescribes context based on data extracted from several databases. Based on this context, the ES 104 then decides on the deployment of the different analysis tools. This is performed by selecting such analysis tools whose pre-defined contextual information is as close as possible to the current prescribed context. In examples, the ES 104 derives a context by comparing predefined context information to current context information extracted from the petrophysical data and drilling data.

In a first example, the current subsurface unit as indicated by the context is extracted from a database, such as a corporate database that includes wellbore metadata interpreted by a geologist. The ES 104 utilizes an aliases system to reconcile the different terms used to refer to the same zone across different disciplines or stakeholders. For example, the petrophysical data and drilling data (e.g., streaming data) is converted to a standardized format in real time. If the ES 104 is unable to extract information or reconcile information, ES 104 predicts the subsurface unit currently being drilled by utilizing a pre-trained Machine Learning model. The ML model that takes as input petrophysical data and well location information to predict the subsurface unit being drilled.

In a second example, the data associated with the hydrocarbon as included in the context is extracted from a database, such as a corporate database that includes wellbore metadata delivered by the asset owner prior to drilling operations beginning at the well. In examples, the hydrocarbon is expected to be found in the data. In examples, there is more than one target hydrocarbon, in which case the asset owner associates each expected subsurface unit with its expected hydrocarbon type. If the ES 104 is unable to extract target hydrocarbon information, the ES predicts the target hydrocarbon of the wellbore by utilizing a pre-trained Machine Learning model. The ML model is a model that takes well location and target subsurface unit information in order to predict what is the target hydrocarbon at the specific subsurface unit.

In a third example, the data associated with the Mud Type/Mud Parameters as included in the context is extracted from a database, such as a corporate database that includes wellbore metadata delivered by the Mud Engineer on site and populated into the database.

In a fourth example, the ES 104 extracts bit size information as included in the context from the WITSML database. This is reliable information automatically populated into the WITSML database serving the real-time petrophysical data.

In a fifth example, the ES 104 extracts service company information as included in the context from the WITSML database. This is reliable information automatically populated into the WITSML database serving the real-time petrophysical data.

The ES 104 prescribes context for each drilled depth. Context can vary as a function of drilled depth as any of the context parameters (such as the five context parameters listed in Table 2) may vary along the drilling operation. With this context information, the ES 104 prescribes specific models to be utilized on all available analysis tools (Table 1). In examples, the ES 104 prescribes models which pre-defined context information is as close as possible to the current context. If the match is not exact, ES generates a warning that alerts the user to a mismatch between the current context and the predefined context information being used. For example, the generated warning specifies the difference between the current prescribed context and the context for which the model was designed to be deployed.

Contexts and other ES prescriptions are displayed in a system 108 that executes a web application for access by the final users. Users with the access privileges can modify the context and prescriptions made by the ES 104 through the web application, overwriting the autonomous decision of the ES, shown by the arrow 130 from the system 108 to the Expert System 104. Upon modification, those new prescriptions are stored by the web application of system 108 in the State Store Module at the state store system 120. Users with access privileges can either change the context, such as the ES estimated context (for example, modifying Target Subsurface Units) or the outcome of the estimated context (for example, choose a different Petrophysical Model, from the list of all Petrophysical Models made available to the platform). The autonomous platform will from then on honor the modifications made by the user for the remaining of the operations in the wellbore where those changes were submitted. Nevertheless, the ES system will continue operating in order to assure autonomy for any parameter not included in the modification made by the user.

The DAS 106 computes the petrophysical output that characterize the wellbores. The DAS 106 receives raw petrophysical data and drilling data, together with instructions from the ES 104. The petrophysical data and drilling data executes those instructions. In examples, the instructions generated by the ES 104 are specific pre-defined models the respective DAS sub-systems (106A, 106B, and 106C) receive as inputs. The DAS is integrated by the following independent, but interoperable sub-systems: formation analysis subsystem 106A, machine learning model deployment subsystem 106B, and specialized calculations subsystem 106C.

In examples, the formation analysis sub-system is a petrophysical solver. Petrophysical solvers are error-minimizing algorithms capable of transforming raw petrophysical measurements into a representation of the formation characteristics nearby the wellbore wall. Examples of formation characteristics are total porosity, water saturation and matrix lithology. In examples, the petrophysical solver is an application written in Python. The petrophysical solver is commercial-grade due to its ability transform any available petrophysical measurement into a representation of the formation characteristics, and to solve non-linear petrophysical models without the need of making simplifying assumptions. In some embodiments, the formation analysis subsystem 106A manages the petrophysical solver, in the context of the autonomous platform.

In examples, the formation analysis subsystem 106A communicates with the Machine Learning Deployment subsystem 106B. For example, if a petrophysical model prescribed by the Expert System (ES) requires petrophysical measurements that are not currently being acquired, the formation analysis subsystem 106A requests that the machine learning deployment subsystem 106B deploy a pre-trained and context-relevant machine learning model available that outputs petrophysical measurements (e.g., curves associated with logged measurements including but not limited to Density, Gamma Ray, and Resistivity logs) that are not currently being acquired. If available, the machine learning deployment subsystem 106B deploys the pre-trained and context-relevant machine learning model prior to the execution of the formation analysis by the formation analysis subsystem 106A, so that the latter can be completed with the relevant information.

In another example, if a machine learning model prescribed by the Expert System (ES) 104 uses petrophysical measurements that are not readily available from raw measurements but computed by the formation analysis subsystem 106A, then the input is obtained from the formation analysis subsystem 106A. In this manner, any outstanding machine learning model can be deployed after to performing formation analysis.

In examples, the machine learning models take as input petrophysical measurements (e.g., petrophysical curves) and output new curves with as many elements as the inputs in a depth-by-depth calculation. For example, inputs to a machine learning model are Density, Gamma-Ray, Resistivity (measurements); example outputs of the machine learning models are Sonic Slowness (this is another measurement, but that is not being measured in this hole, and thus is predicted based on data from nearby wells were this measurement was indeed taken). In another example, inputs to a machine learning model are Density, Porosity, Sonic Slowness (measurements); example outputs of the machine learning model are Rock Strength (Geomechanics parameter). In this example, a model is used to predict a parameter that is measured from an actual rock plug sample at a laboratory. The model is trained from laboratory data, but then deployed at scale as a continuous curve down the hole.

The machine learning deployment subsystem 106B deploys pre-trained machine learning models using raw petrophysical measurements, the output of specialized calculations, and/or the output of the formation analysis subsystem 106A. In examples, a custom deployer tool is used to deploy the machine learning models. The custom deployer is the deployment tool of a software package that is used for training and deploying machine learning models that receive depth-indexed data (e.g., petrophysical data in particular). The custom deployer configured for deployment in an environment where data is obtained in differing formats. Different measurement service providers use different names, sampling rates and corrections for the same physical quantity measured, for example. The custom deployer recognizes the different names, sampling rates, and corrections for the same physical quantity measured, and uses a system of aliases and quality control step to translate and standardize data prior to deployment. In examples, the data is converted to a standardized format prior to being input to the trained machine learning models. The trained models can be deployed on command.

In some embodiments, the machine learning deployment subsystem 106B deploys a set of pre-trained machine learning models as instructed by the Expert System (ES) 104. The outputs (predictions) form part of the set of formation characteristics provided by the autonomous platform system. In examples, the outputs of the petrophysical models are used in other machine learning models to be deployed, formation analysis, and specialized calculations. In examples, the order in which instructions are executed by the sub-systems 126A, 126B, and 126C is determined by the ES 104 so that all inputs/outputs can be utilized efficiently.

In examples, the specialized calculations subsystem 126C is an index of pre-established analytical calculations. The sub-system stores context triggers (Table 2) and maps them with stored analytical calculations. For example, a context trigger can be defined as follows: any wellbore that is being drilled across target subsurface unit: “FOO1”, irrespective of all other 4 contextual parameters must always perform routines “A” & “B”. Routines A & B are stored functionals that can be accessed by the sub-system. These analytical calculations can take as input any form and complexity. The calculations can range from simple arithmetical calculations to complex software. Additionally, the calculations can be any functional that takes inputs and returns outputs. In the autonomous platform, specialized calculations include but are not limited to Geomechanics, Laminated Shale Analysis, Mud-Logging Analysis, NMR Porosimetry Analysis, NMR Heavy Oil Analysis, Density Image Automatic Interpretation, Resistivity Image Automatic Interpretation, Automatic Bed-Boundary detection, or any combinations thereof. Although particular calculations are described as specialized calculations, any predetermined calculations can be used. For example, specialized calculations are selected based on the data that is available to be further analyzed at the particular wellbore, and also on the specific objectives of the borehole in question. The ES 104 uses the available data and specific objectives, and translates context (wellbore location, targeted geological formation, among others) into the set of specialized calculations. In real time, the system determines whether or not it can supply the needed inputs based on what the data that is available at the time of drilling.

The outputs of the specialized calculation subsystem 106C form part of the formation characteristics delivered in real-time by the autonomous platform. In examples, the specialized calculations subsystem 106C is implemented through Physical Models.

In the example of FIG. 1, the State Store System (SSS) 120 is a relational database that stores the most current snapshot of the state of the platform at any given time. Specifically, it stores: (i) data acquired by the DC&ES 102; (ii) Data output by the DAS sub-systems 106A-106C; (iii) Context estimates made by the ES 104 and the parameters delivered to the DAS sub-systems; and (iv) User-Defined context and parameters prescribed through the Web Application interface (shown by arrow 128 of FIG. 1).

Error! Reference source not found. shows a workflow 200 between a State Store System (SSS) 220 and a Data Consultation and Extraction System (DC&ES) 202. In examples. The DC&ES 202, application 202A, application 202B, expert system 204, and state store system 220 are the same as or similar to the DC&ES 102, application 102A, application 102B, expert system 104, and state store system 120 of FIG. 1.

In some embodiments, the DC&ES 202 makes frequent requests for data headers present in the WITSML database. At reference number 242, the DC&ES 202 consults headers of active wellbores. Headers store a description of the data present in a respective database. The headers are of a small size such that the request to determine data present in a respective database using the header is low-latency, allowing for frequent queries in real time. The header information is compared to the state of the system in the SSS 220. At reference number 244, a determination is made on if the headers describe data that is not already stored by the SSS 220. If the queried headers indicate there is data present in the respective database that has not been stored in the SSS 220, the state system prescribes the wellbores, channels, and depth intervals associated with the missing data as described by the headers at reference number 246. The missing data is likely due to the data being acquired since the last request for header data. In response to the data present in the respective database that has not been stored in the SSS 220, a query is prepared for the actual data missing at reference number 248. In examples, the query includes the channels and depth intervals of specific wellbores. This enables the system to be memory efficient, lean and responsive. The data extracted is appended to the SSS 220 at reference number 250, and also transmitted to the ES 204. Once received by the ES 204, a Data Analysis System (DAS) obtains the new data as shown at reference number 252. The newly extracted, usually short depth interval of data is analyzed, which provides fast responsiveness and formation characteristics once received.

In examples, the output of the DAS for this newly acquire interval is appended to the SSS 220, so that the SSS 220 includes the most up to date state of the system. For example, as shown in FIG. 1, arrow 134 shows the output of the DAS for a newly acquire interval being transmitted to the SSS 120. Referring again to FIG. 1, the system 108 is a visualization and supervision system (VSS). The fundamental objective of the VSS 108 is twofold. Firstly, the VSS 108 continuously displays the data available in the SSS 120 to the multiple end users over a network. The VSS 108 contains dashboards and layouts that render representations petrophysical data and drilling data in a clear and informative way. These visualization tools are interactive and enable a user to take rapid action based on the data rendered at the display associated with the VSS 108. Additionally, the VSS 108 enables a user to export the data so that it can be further analyzed by external tools if necessary. Secondly, the VSS 108 enables the end user to overwrite the expert system 104 and customize the way the data is analyzed. These instructions are passed to the ES 104 and are enforced for the rest of the data acquisition operation of the specific wellbore in question. In examples, the VSS 108 is implemented by creating a web application called WebAPP that connects to the SSS relational database and displays its data. WebAPP makes high frequency and periodic queries to the SSS database to ensure it is always displaying quasi-instantaneous snapshot of the state of the system.

In examples, the results of the real-time formation evaluation by the DAS 106 are a set of curves describing the volumetrics of the formation, and the VSS visualizes the volumetrics. The volumetrics characterize (i) the matrix composition (e.g., solid rock, inclusive of water bound to clay minerals); (ii) the pore spaces (e.g., how much volume is void of solid rock and water bound to clay minerals); and (iii) the fluid composition (e.g., which fluids fill the aforementioned pore spaces). Examples of each respective volumetrics category are: (i) The volume of Quartz, Illite or Calcite; (ii) PHIT (Total Porosity); and (iii) Volume of Oil, Gas or Water. These curves describing the volumetrics have the same length as the length of the input curves (e.g., petrophysical measurements) to the DAS 106. Thus, if at any given time, measurements such as Density, Resistivity and Gamma Ray have been acquired from depth 5,000′ to depth 6,000′ and sampled every 1 foot, then those curves have 1,001 data points each. When analyzed by the formation evaluation sub-system 106A, it will output curves describing the volumetrics with 1,001 points each as well. In other words, the analysis is performed per-depth.

The specific volumetric curves the formation evaluation sub-system 106A outputs is dependent on the petrophysical model prescribed for the analysis of that specific formation. For example, a petrophysical model may call to solve for the volume of Calcite, Dolomite, Porosity, Water and Gas, while another one may call to solve for volume of Quartz, Illite, Porosity, Water and Oil. In examples, porosity and water are determined by the petrophysical solver.

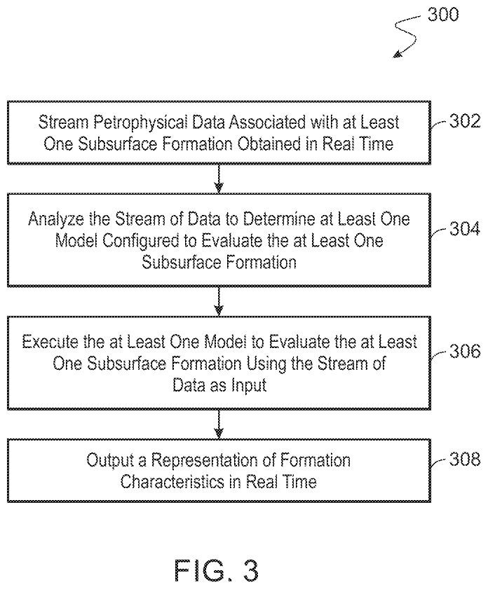

FIG. 3 is a process flow diagram of a process 300 that enables real-time and autonomous petrophysical formation evaluation and machine learning deployment.

At block 302, streaming data including petrophysical data associated with at least one subsurface formation is obtained in real-time. In examples, petrophysical data and drilling data associated with at least one subsurface formation are obtained in real-time.

At block 304, the streaming data is continuously analyzed to deduce models/parameters that are executed to evaluate a respective subsurface formation in view of a context of the evaluation.

At block 306, execution of the models/parameters in view of the context occurs in a sequence based on the available streaming data (e.g., the Machine Learning Deployment sub-system deploys that ML model previous to the Formation Analysis, so that the latter can be completed).

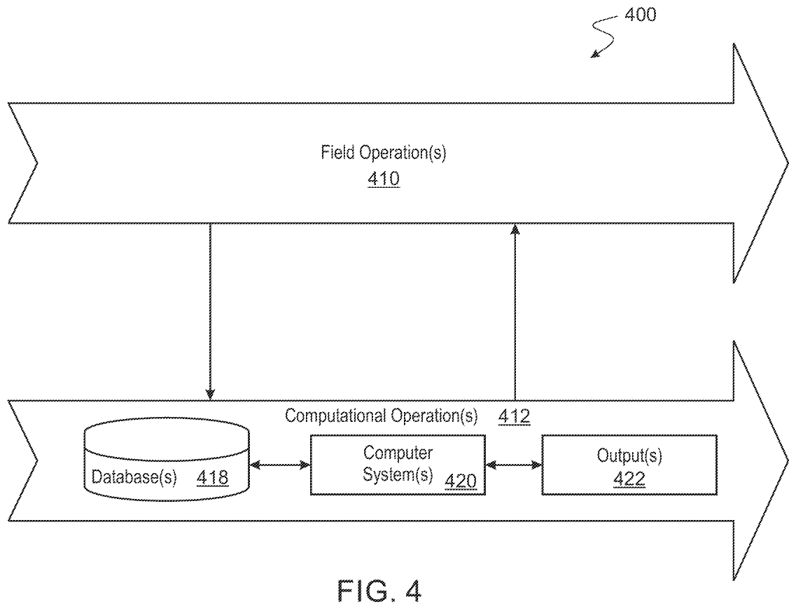

At block 308, a formation characterization is output in real time by execution of the models/parameters. In examples, outputting the formation characterization in real time comprises rendering the formation characterization for multiple instances of a visualization system. Additionally, in examples, the formation characterization is used in geosteering operations, formation evaluation, completions placement, drilling optimization, core/casing point selection, formation testing/sampling design, or any combinations thereof. In an example, formation characterization is used in geosteering operations, where the borehole position (inclination and azimuth angles) is adjusted on the fly to reach one or more geological targets during drilling. These changes are based on formation characterization determined in real time while drilling. In an example, the formation characterization is used to guide hydrocarbon production operations as described with respect to FIG. 4.

FIG. 4 illustrates hydrocarbon production operations 400 that include both one or more field operations 410 and one or more computational operations 412, which exchange information and control exploration for the production of hydrocarbons. In some implementations, outputs of techniques of the present disclosure can be performed before, during, or in combination with the hydrocarbon production operations 400, specifically, for example, either as field operations 410 or computational operations 412, or both.

Examples of field operations 410 include forming/drilling a wellbore, hydraulic fracturing, producing through the wellbore, injecting fluids (such as water) through the wellbore, to name a few. In some implementations, methods of the present disclosure can trigger or control the field operations 410. For example, the methods of the present disclosure can generate data from hardware/software including sensors and physical data gathering equipment (e.g., seismic sensors, well logging tools, flow meters, and temperature and pressure sensors). The methods of the present disclosure can include transmitting the data from the hardware/software to the field operations 410 and responsively triggering the field operations 410 including, for example, generating plans and signals that provide feedback to and control physical components of the field operations 410. Alternatively or in addition, the field operations 410 can trigger the methods of the present disclosure. For example, implementing physical components (including, for example, hardware, such as sensors) deployed in the field operations 410 can generate plans and signals that can be provided as input or feedback (or both) to the methods of the present disclosure.

Examples of computational operations 412 include one or more computer systems 420 that include one or more processors and computer-readable media (e.g., non-transitory computer-readable media) operatively coupled to the one or more processors to execute computer operations to perform the methods of the present disclosure. The computational operations 412 can be implemented using one or more databases 418, which store data received from the field operations 410 and/or generated internally within the computational operations 412 (e.g., by implementing the methods of the present disclosure) or both. For example, the one or more computer systems 420 process inputs from the field operations 410 to assess conditions in the physical world, the outputs of which are stored in the databases 418. For example, seismic sensors of the field operations 410 can be used to perform a seismic survey to map subterranean features, such as facies and faults. In performing a seismic survey, seismic sources (e.g., seismic vibrators or explosions) generate seismic waves that propagate in the earth and seismic receivers (e.g., geophones) measure reflections generated as the seismic waves interact with boundaries between layers of a subsurface formation. The source and received signals are provided to the computational operations 412 where they are stored in the databases 418 and analyzed by the one or more computer systems 420.

In some implementations, one or more outputs 422 generated by the one or more computer systems 420 can be provided as feedback/input to the field operations 410 (either as direct input or stored in the databases 418). The field operations 410 can use the feedback/input to control physical components used to perform the field operations 410 in the real world.

For example, the computational operations 412 can process the seismic data to generate three-dimensional (3D) maps of the subsurface formation. The computational operations 412 can use these 3D maps to provide plans for locating and drilling exploratory wells. In some operations, the exploratory wells are drilled using logging-while-drilling (LWD) techniques which incorporate logging tools into the drill string. LWD techniques can enable the computational operations 412 to process new information about the formation and control the drilling to adjust to the observed conditions in real-time.

The one or more computer systems 420 can update the 3D maps of the subsurface formation as information from one exploration well is received and the computational operations 412 can adjust the location of the next exploration well based on the updated 3D maps. Similarly, the data received from production operations can be used by the computational operations 412 to control components of the production operations. For example, production well and pipeline data can be analyzed to predict slugging in pipelines leading to a refinery and the computational operations 412 can control machine operated valves upstream of the refinery to reduce the likelihood of plant disruptions that run the risk of taking the plant offline.

In some implementations of the computational operations 412, customized user interfaces can present intermediate or final results of the above-described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or app), or at a central processing facility.

The presented information can include feedback, such as changes in parameters or processing inputs, that the user can select to improve a production environment, such as in the exploration, production, and/or testing of petrochemical processes or facilities. For example, the feedback can include parameters that, when selected by the user, can cause a change to, or an improvement in, drilling parameters (including drill bit speed and direction) or overall production of a gas or oil well. The feedback, when implemented by the user, can improve the speed and accuracy of calculations, streamline processes, improve models, and solve problems related to efficiency, performance, safety, reliability, costs, downtime, and the need for manual interpretation.

In some implementations, the feedback can be implemented in real-time, such as to provide an immediate or near-immediate change in operations or in a model. The term real-time (or similar terms as understood by one of ordinary skill in the art) means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second(s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

Events can include readings or measurements captured by downhole equipment such as sensors, pumps, bottom hole assemblies, or other equipment. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas well exploration, production/drilling, or testing. For example, outputs of the present disclosure can be used as inputs to other equipment and/or systems at a facility. This can be especially useful for systems or various pieces of equipment that are located several meters or several miles apart, or are located in different countries or other jurisdictions.

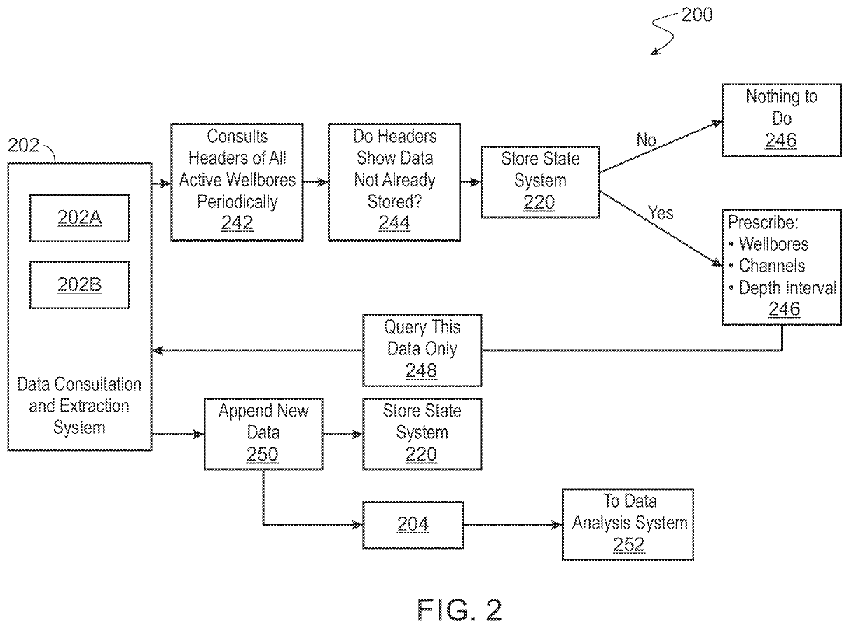

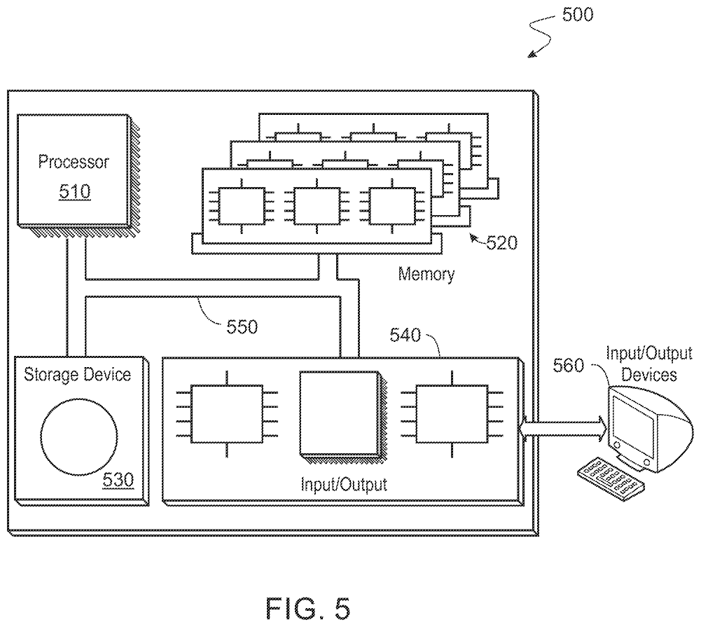

FIG. 5 is a schematic illustration of an example controller 500 (or control system) for that enables real-time and autonomous petrophysical formation evaluation and machine learning deployment. For example, the controller 500 may be operable according to the workflow 100 of FIG. 1 or the workflow 200 of FIG. 2. In some embodiments, the controller 500 is the same as or similar to the computer systems 420 of FIG. 4. The controller 500 is intended to include various forms of digital computers, such as printed circuit boards (PCB), processors, digital circuitry, or otherwise parts of a system for supply chain alert management. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 500 includes a processor 510, a memory 520, a storage device 530, and an input/output interface 540 communicatively coupled with input/output devices 560 (for example, displays, keyboards, measurement devices, sensors, valves, pumps). Each of the components 510, 520, 530, and 540 are interconnected using a system bus 550. The processor 510 is capable of processing instructions for execution within the controller 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output interface 540.

The memory 520 stores information within the controller 500. In one implementation, the memory 520 is a computer-readable medium. In one implementation, the memory 520 is a volatile memory unit. In another implementation, the memory 520 is a nonvolatile memory unit.

The storage device 530 is capable of providing mass storage for the controller 500. In one implementation, the storage device 530 is a computer-readable medium. In various different implementations, the storage device 530 may be a floppy disk device, a hard disk device, an optical disk device, or a tape device.

The input/output interface 540 provides input/output operations for the controller 500. In one implementation, the input/output devices 560 includes a keyboard and/or pointing device. In another implementation, the input/output devices 560 includes a display unit for displaying graphical user interfaces.

There can be any number of controllers 500 associated with, or external to, a computer system containing controller 500, with each controller 500 communicating over a network. Further, the terms “client,” “user,” and other appropriate terminology can be used interchangeably, as appropriate, without departing from the scope of the present disclosure. Moreover, the present disclosure contemplates that many users can use one controller 500 and one user can use multiple controllers 500.

According to some non-limiting embodiments or examples, provided is a computer-implemented method that enables real time and autonomous petrophysical formation evaluation and machine learning deployment, comprising: streaming, using at least one hardware processor, data comprising petrophysical data associated with at least one subsurface formation obtained in real time; analyzing, using the at least one hardware processor, the stream of data to determine at least one model configured to evaluate the at least one subsurface formation; executing, using the at least one hardware processor, the at least one model to evaluate the at least one subsurface formation using the stream of data as input; and outputting, using the at least one hardware processor, a representation of formation characteristics in real time.

According to some non-limiting embodiments or examples, provided is a system, comprising: at least one processor, and at least one non-transitory storage media storing instructions that, when executed by the at least one processor, cause the at least one processor to: stream data comprising petrophysical data associated with at least one subsurface formation obtained in real time; analyze the stream of data to determine at least one model configured to evaluate the at least one subsurface formation; execute the at least one model to evaluate the at least one subsurface formation using the stream of data as input; and output a representation of formation characteristics in real time.

According to some non-limiting embodiments or examples, provided is at least one non-transitory storage media storing instructions that, when executed by at least one processor, cause the at least one processor to: stream data comprising petrophysical data associated with at least one subsurface formation obtained in real time; analyze the stream of data to determine at least one model configured to evaluate the at least one subsurface formation; execute the at least one model to evaluate the at least one subsurface formation using the stream of data as input; and output a representation of formation characteristics in real time.

Further non-limiting aspects or embodiments are set forth in the following numbered embodiments:

Embodiment 1: A computer-implemented method that enables real time and autonomous petrophysical formation evaluation and machine learning deployment, comprising: streaming, using at least one hardware processor, data comprising petrophysical data associated with at least one subsurface formation obtained in real time; analyzing, using the at least one hardware processor, the stream of data to determine at least one model configured to evaluate the at least one subsurface formation; executing, using the at least one hardware processor, the at least one model to evaluate the at least one subsurface formation using the stream of data as input; and outputting, using the at least one hardware processor, a representation of formation characteristics in real time.

Embodiment 2: The computer implemented method of embodiment 1, comprising analyzing the stream of data to determine models configured to simultaneously evaluate data associated with multiple subsurface formations obtained in real time

Embodiment 3: The computer implemented method of embodiments 1 or 2, wherein outputting the formation characterization in real time comprises rendering the formation characterization for multiple instances of a visualization system.

Embodiment 4: The computer implemented method of embodiments 1-3, wherein the stream of data is converted to a standardized format in real time.

Embodiment 5: The computer implemented method of embodiments 1-4, comprising analyzing the stream of data to determine at least one model configured to evaluate the at least one subsurface formation in view of a context extracted from the petrophysical data and drilling data.

Embodiment 6: The computer implemented method of embodiments 1-5, wherein the at least one model is a trained machine learning model deployed based on a type of inputs to the trained machine learning model being found in the petrophysical data.

Embodiment 7: The computer implemented method of embodiments 1-6, wherein the at least one model is deployed using a custom deployer configured for deployment in an environment where data is obtained in differing formats.

Embodiment 8: A system, comprising: at least one processor, and at least one non-transitory storage media storing instructions that, when executed by the at least one processor, cause the at least one processor to: stream data comprising petrophysical data associated with at least one subsurface formation obtained in real time; analyze the stream of data to determine at least one model configured to evaluate the at least one subsurface formation; execute the at least one model to evaluate the at least one subsurface formation using the stream of data as input; and output a representation of formation characteristics in real time.

Embodiment 9: The system of embodiment 8, comprising analyzing the stream of data to determine models configured to simultaneously evaluate data associated with multiple subsurface formations obtained in real time

Embodiment 10: The system of embodiments 8 or 9, wherein outputting the formation characterization in real time comprises rendering the formation characterization for multiple instances of a visualization system.

Embodiment 11: The system of embodiments 8-10, wherein the stream of data is converted to a standardized format in real time.

Embodiment 12: The system of embodiments 8-11, comprising analyzing the stream of data to determine at least one model configured to evaluate the at least one subsurface formation in view of a context extracted from the petrophysical data and drilling data.

Embodiment 13: The system of embodiments 8-12, wherein the at least one model is a trained machine learning model deployed based on a type of inputs to the trained machine learning model being found in the petrophysical data.

Embodiment 14: The system of embodiments 8-13, wherein the at least one model is deployed using a custom deployer configured for deployment in an environment where data is obtained in differing formats.

Embodiment 15: At least one non-transitory storage media storing instructions that, when executed by at least one processor, cause the at least one processor to: stream data comprising petrophysical data associated with at least one subsurface formation obtained in real time; analyze the stream of data to determine at least one model configured to evaluate the at least one subsurface formation; execute the at least one model to evaluate the at least one subsurface formation using the stream of data as input; and output a representation of formation characteristics in real time.

Embodiment 16: The at least one non-transitory storage media of embodiment 15, comprising analyzing the stream of data to determine models configured to simultaneously evaluate data associated with multiple subsurface formations obtained in real time.

Embodiment 17: The at least one non-transitory storage media of embodiments 15 or 16, wherein outputting the formation characterization in real time comprises rendering the formation characterization for multiple instances of a visualization system.

Embodiment 18: The at least one non-transitory storage media of embodiments 15-17, wherein the stream of data is converted to a standardized format in real time.

Embodiment 19: The at least one non-transitory storage media of embodiments 15-18, comprising analyzing the stream of data to determine at least one model configured to evaluate the at least one subsurface formation in view of a context extracted from the petrophysical data and drilling data.

Embodiment 20: The at least one non-transitory storage media of embodiments 15-19, wherein the at least one model is a trained machine learning model deployed based on a type of inputs to the trained machine learning model being found in the petrophysical data.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example, LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that is used by the user. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back end component, for example, as a data server, or that includes a middleware component, for example, an application server. Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship. Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

What is claimed is:

1. A computer-implemented method that enables real time and autonomous petrophysical formation evaluation and machine learning deployment, comprising:
streaming, using at least one hardware processor, data comprising petrophysical data associated with at least one subsurface formation obtained in real time;
analyzing, using the at least one hardware processor, the stream of data to determine at least one model configured to evaluate the at least one subsurface formation;
executing, using the at least one hardware processor, the at least one model to evaluate the at least one subsurface formation using the stream of data as input; and
outputting, using the at least one hardware processor, a representation of formation characteristics in real time.

2. The computer implemented method of claim 1, comprising analyzing the stream of data to determine models configured to simultaneously evaluate data associated with multiple subsurface formations obtained in real time.

3. The computer implemented method of claim 1, wherein outputting the formation characterization in real time comprises rendering the formation characterization for multiple instances of a visualization system.

4. The computer implemented method of claim 1, wherein the stream of data is converted to a standardized format in real time.

5. The computer implemented method of claim 1, comprising analyzing the stream of data to determine at least one model configured to evaluate the at least one subsurface formation in view of a context extracted from the petrophysical data and drilling data.

6. The computer implemented method of claim 1, wherein the at least one model is a trained machine learning model deployed based on a type of inputs to the trained machine learning model being found in the petrophysical data.

7. The computer implemented method of claim 1, wherein the at least one model is deployed using a custom deployer configured for deployment in an environment where data is obtained in differing formats.

8. A system, comprising:
at least one processor, and
at least one non-transitory storage media storing instructions that, when executed by the at least one processor, cause the at least one processor to:
stream data comprising petrophysical data associated with at least one subsurface formation obtained in real time;
analyze the stream of data to determine at least one model configured to evaluate the at least one subsurface formation;
execute the at least one model to evaluate the at least one subsurface formation using the stream of data as input; and
output a representation of formation characteristics in real time.

9. The system of claim 8, comprising analyzing the stream of data to determine models configured to simultaneously evaluate data associated with multiple subsurface formations obtained in real time.

10. The system of claim 8, wherein outputting the formation characterization in real time comprises rendering the formation characterization for multiple instances of a visualization system.

11. The system of claim 8, wherein the stream of data is converted to a standardized format in real time.

12. The system of claim 8, comprising analyzing the stream of data to determine at least one model configured to evaluate the at least one subsurface formation in view of a context extracted from the petrophysical data and drilling data.

13. The system of claim 8, wherein the at least one model is a trained machine learning model deployed based on a type of inputs to the trained machine learning model being found in the petrophysical data.

14. The system of claim 8, wherein the at least one model is deployed using a custom deployer configured for deployment in an environment where data is obtained in differing formats.

15. At least one non-transitory storage media storing instructions that, when executed by at least one processor, cause the at least one processor to:
stream data comprising petrophysical data associated with at least one subsurface formation obtained in real time;
analyze the stream of data to determine at least one model configured to evaluate the at least one subsurface formation;
execute the at least one model to evaluate the at least one subsurface formation using the stream of data as input; and
output a representation of formation characteristics in real time.

16. The at least one non-transitory storage media of claim 15, comprising analyzing the stream of data to determine models configured to simultaneously evaluate data associated with multiple subsurface formations obtained in real time.

17. The at least one non-transitory storage media of claim 15, wherein outputting the formation characterization in real time comprises rendering the formation characterization for multiple instances of a visualization system.

18. The at least one non-transitory storage media of claim 15, wherein the stream of data is converted to a standardized format in real time.

19. The at least one non-transitory storage media of claim 15, comprising analyzing the stream of data to determine at least one model configured to evaluate the at least one subsurface formation in view of a context extracted from the petrophysical data and drilling data.

20. The at least one non-transitory storage media of claim 15, wherein the at least one model is a trained machine learning model deployed based on a type of inputs to the trained machine learning model being found in the petrophysical data.
